(19) United States
(12) Patent Application Publication
(10) Pub. No.: US 2023/0321489 A1
(43) Pub. Date: Oct. 12, 2023

(54) SYSTEM AND TECHNIQUES FOR PROVIDING CONTINUOUS AUDITORY FEEDBACK FOR PERFORMANCE-BASED ACTIVITY

(71) Applicant: Rush University Medical Center, Chicago, IL (US)

(72) Inventors: Markus WIMMER, Chicago, IL (US); Luisa CEDIN, Aurora, IL (US); Christopher KNOWLTON, Chicago, IL (US)

(73) Assignee: Rush University Medical Center, Chicago, IL (US)

(21) Appl. No.: 18/131,849

(22) Filed: Apr. 6, 2023

Related U.S. Application Data
(60) Provisional application No. 63/328,424, filed on Apr. 7, 2022.

(51) Int. Cl. A63B 24/00; G16H 40/67; G16H 20/30

(52) U.S. Cl. CPC A63B 24/0062; G16H 40/67; G16H 20/30; A63B 2024/0068; A63B 2071/0625

(57) ABSTRACT

An aspect of the present disclosure describes a system for providing continuous and progressive auditory feedback based on real-time orthopedic sensor data, which includes a plurality of plantar pressure sensors, an audio output device, and a mobile device. The mobile device receives receive, from at least one of the plantar pressure sensors, one or more sensor values indicative of an actual movement of a user during a training session. The mobile device determines, based on an evaluation of the sensor values, a similarity of the actual movement to a target movement. The mobile device modifies, as a function of the similarity of the actual movement to the target movement, an auditory feedback, and causes the audio output device to output the auditory feedback.

### CROSS-REFERENCE TO RELATED APPLICATIONS

This application claims the benefit of U.S. Provisional Patent Application No. 63/328,424, filed Apr. 7, 2022, which is expressly incorporated by reference and made a part hereof.

### TECHNICAL FIELD

The present disclosure generally relates to movement sonification, and more particularly, to techniques for generating and manipulating auditory feedback output in response to observed plantar sensor data.

### BACKGROUND

Movement sonification generally pertains to using non-speech audio to convey information pertaining to an observed movement. Sonification has clinical applications in physical rehabilitation, in which a patient performs certain movements and receives auditory signals based on observed movements. For example, in orthopedics, plantar pressure sensors may be outfitted in a patient's shoes and capture plantar pressure data as the patient performs walking movements, which the sensors can then transmit to a system. The system is able to generate, based on the plantar pressure data, analytics regarding various characteristics of the patient's gait, such as foot roll over, pressure applied towards various portions of each foot during a step, and the like. Based on such analytics, the system outputs audio that indicative of the movement. Using sonification in the rehabilitation setting can allow the patient to use the audio as a cue to adjust subsequent movements to better work towards a target baseline.

However, current approaches towards sonifying user movement (e.g., a patient movement) are primitive and typically drawn to negative stimuli. Continuing the example above, a patient might receive auditory feedback, such as a short tone or buzz, in instances that the patient is applying too much or too little pressure on a given portion of the foot relative to a baseline pressure. Such negative stimuli can discourage a patient from continuing to seek treatment using sonification.

The description provided in the background section should not be assumed to be prior art merely because it is mentioned in or associated with the background section. The background section may include information that describes one or more aspects of the subject technology.

### SUMMARY

One embodiment presented herein discloses a system. The system includes a plurality of plantar pressure sensors, an audio output device, and a mobile device. The mobile device comprises a processor and a plurality of instructions. The plurality of instructions, when executed by the processor, cause the mobile device to receive, from at least one of the plantar pressure sensors, one or more sensor values. The sensor values are indicative of an actual movement of a user during a training session. The plurality of instructions cause the mobile device to determine, based on an evaluation of the sensor values, a similarity of the actual movement to a target movement. The mobile device modifies, as a function of the similarity of the actual movement to the target movement, an auditory feedback to be output. The mobile device causes the audio output device to output the auditory feedback.

Another embodiment presented herein discloses a method. The method generally includes receiving, one at least one of a plurality of plantar pressure sensors, one or more sensor values indicative of an actual movement of a user during a training session. The method also generally includes determining, based on an evaluation of the sensor values, a similarity of the actual movement to a target movement. An auditory feedback to be output is modified as a function of the similarity of the actual movement to the target movement. The method also generally includes causing an audio output device to output the auditory feedback.

Yet another embodiment presented herein discloses a computer-readable storage medium storing a plurality of instructions. The plurality of instructions, when executed by a processor, causes a mobile device to receive, from at least one of a plurality of plantar pressure sensors, one or more sensor values. The sensor values are indicative of an actual movement of a user during a training session. The plurality of instructions cause the mobile device to determine, based on an evaluation of the sensor values, a similarity of the actual movement to a target movement. The mobile device modifies, as a function of the similarity of the actual movement to the target movement, an auditory feedback to be output. The mobile device causes an audio output device to output the auditory feedback.

Other aspects and advantages of the present disclosure will become apparent upon consideration of the following detailed description and the attached drawings wherein like numerals designate like structures throughout the specification.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments.

FIG. 1 illustrates a conceptual diagram of an example computing environment in which a computing device provides continuous and progressive auditory feedback based on real-time orthopedic sensor data, according to an embodiment;

FIG. 2 illustrates a block diagram of an example mobile device configured to provide continuous and progressive auditory feedback based on real-time orthopedic sensor data, according to an embodiment;

FIG. 3 illustrates a conceptual diagram of an example operating environment established by an application configured to provide continuous and progressive auditory feedback based on real-time orthopedic sensor data, according to an embodiment;

FIG. 4 illustrates a flow diagram of an example method for initializing one or more devices for providing continuous and progressive auditory feedback based on real-time orthopedic sensor data, according to an embodiment;

FIG. 5 illustrates a flow diagram of an example method for providing continuous and progressive auditory feedback based on real-time orthopedic sensor data, according to an embodiment;

FIGS. 6A-6C illustrate example diagrams of a baseline data collection of a walk captured by one or more real-time orthopedic sensors, according to an embodiment; and

FIGS. 7A-7C illustrate example diagrams of a patient data collection of a walk captured by one or more real-time orthopedic sensors, in which the patient received continuous and progressive auditory feedback according to the embodiments described herein.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

Embodiments presented herein disclose a system and techniques for providing continuous and progressive auditory feedback based on real-time orthopedic sensor data. The system and techniques of the present disclosure will provide a user, such as an orthopedic patient or an athlete, an interactive approach for optimizing user movement for rehabilitation or performance. As further described herein, the embodiments may be adapted to a mobile device and sensor arrangement, in which a mobile device application (“app”) receives data over a wireless connection from plantar sensor insoles placed within shoes of the user. Based on the data received, the mobile device app is able to identify various characteristics of a user gait, such as plantar pressure in various portions of each foot, acceleration, total force, rotation, elapsed time and step count, and the like. Based on these characteristics, the app generates or modifies auditory feedback for output by the mobile device.

The app, in response to user movements that are closely similar relative to a target movement, may generate auditory feedback that builds upon previously generated feedback to provide affirmative encouragement to the user. For example, some auditory feedback may correspond to a music track having audio channels that are introduced in sequence as the user continues to move in accordance to a target. As another example, some auditory feedback may correspond to a chord progression, in which the app generates and outputs chords in sequence as the user continues to move in accordance to the target.

As yet another example, the app may modify auditory feedback currently being output in response to user movement that deviates from a target. For instance, given the music track discussed above, the mobile device may deactivate or muffle certain audio channels in response to user movement that deviates from the target. Given the chord progression discussed above, the mobile device may generate a chord that falls outside of the current progression in response to a deviating user movement.

Advantageously, the system and techniques disclosed herein provide a reward-based and interactive approach that continues to generate progressive feedback in response to various stimuli associated with user movement. By rewarding user movement that closely conforms with a target (and guiding user movement that deviates therefrom), the user may be further encouraged to engage with the system, e.g., to monitor, modify, and make tangible plantar pressure by listening to the auditory feedback. In addition, as further described herein, clinical experiments using the techniques described herein have demonstrated an improvement in a gait of a patient undergoing orthopedic rehabilitation.

Although tangible benefits in rehabilitation are obtainable using the embodiments of the present disclosure, such techniques may also be applied to other settings, such as sports performance. For example, the app may orient the user to perform a run while matching a target running cadence based on beats per minute of a given audio track. The app may modify the audio track as the user progresses through the run and matches or deviates from the target running cadence. Further, one of skill in the art will recognize that while walking and running are reference examples of providing continuous and progressive real-time auditory feedback, such techniques may be adapted other physical movement activity such as climbing, squat exercises, and balance training.

The detailed description set forth herein is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

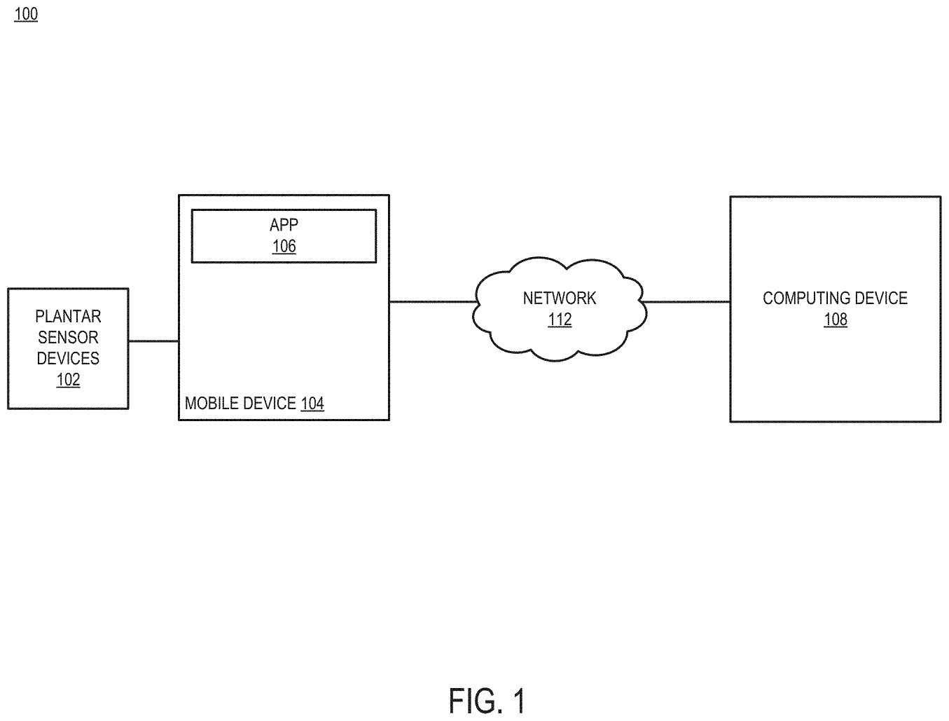

Turning now to FIG. 1, an example computing environment 100 is shown. Illustratively, the environment 100 includes one or more plantar sensor devices 102, a mobile device 104, a computing device 108, and a network 112.

Each plantar sensor device 102 may be embodied as an insole that is inserted into a shoe of the user. The insole includes a number of individual plantar pressure sensors that are positioned to receive and measure pressure from a given region of the foot. The plantar sensor device 102 may also include components such as an inertial measurement unit (IMU) for measuring specific force, angular rate, orientation, and so on, associated with the insole as the user performs a movement. In this example, each plantar sensor device 102 includes a sixteen plantar sensors and a six-axis IMU. Of course, other configurations may be contemplated.

Further, each plantar sensor device 102 may include network interface components that allow coupling to a computing device, such as the mobile device 104, over a wired or wireless network connection, such as a connection over BLUETOOTH or WIFI. In addition, the plantar sensor device 102 may implement a network communication protocol to enable the plantar sensor device 102 to continuously transmit data captured by the sensors and IMU once a network connection has been established. Example network communication protocols that may be implemented are User Datagram Protocol (UDP), Wireless Application Protocol (WAP), and Transmission Control Protocol (TCP).

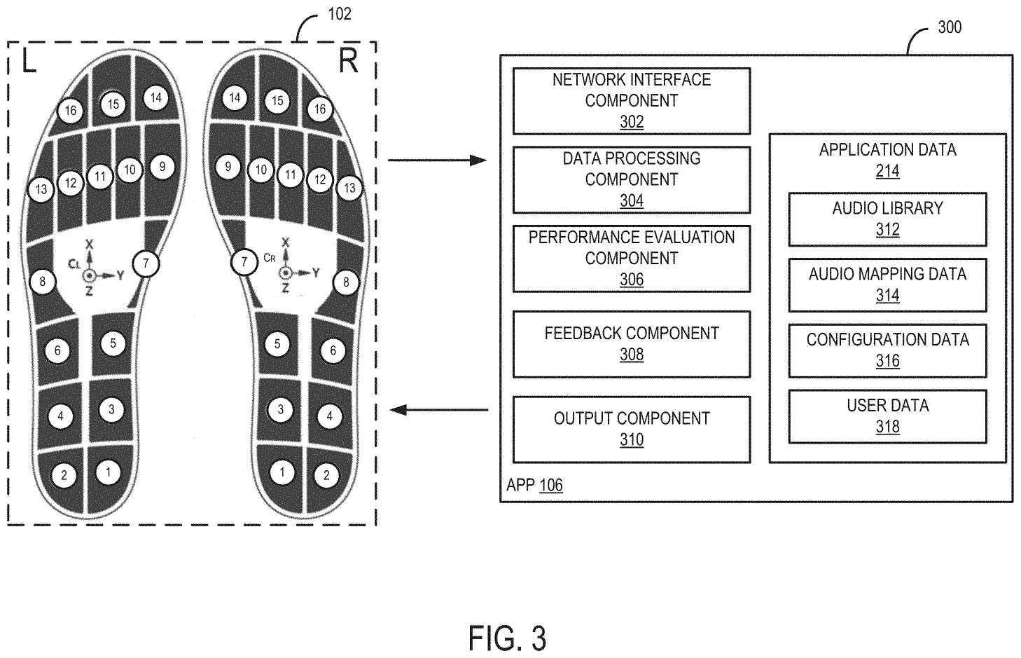

In an embodiment, the mobile device 104 is a smartphone device that the user might possess while engaging with the plantar sensor devices 102. Other examples of a mobile device 104 that may be adapted are tablet device and wearable devices such as smartwatches. As shown, the mobile device 104 includes an app 106, which, in execution, is configured to perform the techniques described herein. More particularly, the app 106 may be configured to establish a network connection with the plantar sensor devices 102, receive data from the plantar sensor devices 102, and generate and modify auditory feedback for output based on the received data. Operational functions of the app 104 are explained in further detail relative to FIG. 3. In an embodiment, the app 106 may use sensors within the mobile device 104, such as an internal accelerometer, gyroscope, and any other sensors capturing movement thereof (and by proxy, the user) may be used in addition to (or in replacement of) one or more of the plantar sensor devices 102 for a more robust and accurate approach to ascertaining user movements.

In an embodiment, the app 106 may receive application data from (e.g., usage data, audio library files, configuration data, analytics, etc.) and transmit application data to the c 108 over the network 112. The computing device 108 may be a physical computing system, a virtual computing instance executing in the cloud, a rack server, and the like. The computing device 108 may store application data and also conduct further analytics associated with data transmitted by the app 106. The network 112 may be embodied as any computer network in which the mobile device 104 and the computing device 108 may communicate, such as a local area network, a wide area network, or the Internet.

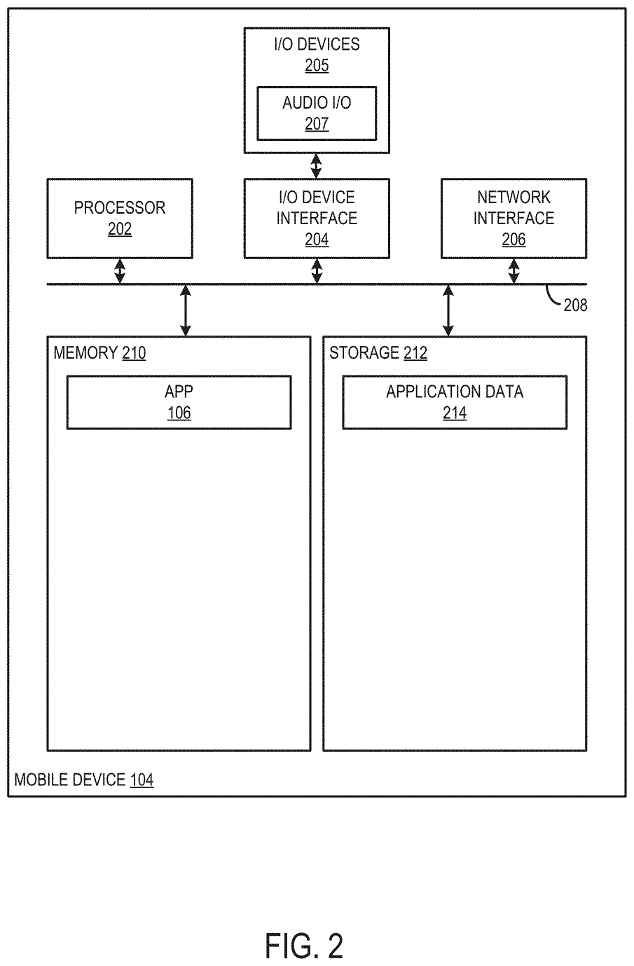

Referring now to FIG. 2, components of the mobile device 104 are shown in more detail. As shown, the mobile device 104 includes, without limitation, one or more processors 202, an I/O device interface 204, I/O devices 205, a network interface 206, a memory 210, and a storage 212, each interconnected via a hardware bus 208. Of course, the actual mobile device 104 will include a variety of additional hardware (or software-based) components not shown (including the internal accelerometer and gyroscope sensors described above). Additionally, in some embodiments, one or more of the illustrative components may be incorporated in, or otherwise form a portion of, another component.

The processor 202 retrieves and executes programming instructions stored in the memory 210 capable of performing the functions described herein. The processor 202 may be embodied as a single or multi-core processor(s), a graphics processor, a microcontroller, or other processor or processing/controlling circuit. In some embodiments, the processor 202 may be embodied as, include, or be coupled to a field programmable gate array (FPGA), an application-specific integrated circuit (ASIC), reconfigurable hardware or hardware circuitry, or other specialized hardware to enable performance of the functions described herein. The hardware bus 208 is used to transmit instructions and data between the processor 202, storage 212, network interface 206, and the memory 210. The memory 210 may be embodied as any type of volatile (e.g., dynamic random access memory, etc.) or non-volatile memory (e.g., byte addressable memory) or data storage capable of performing the functions described herein. Volatile memory may be a storage medium that requires power to maintain the state of data stored by the medium. Non-limiting examples of volatile memory may include various types of random access memory (RAM), such as DRAM or static random access memory (SRAM). One particular type of DRAM that may be used in a memory module is synchronous dynamic random access memory (SDRAM). In particular embodiments, DRAM of a memory component may comply with a standard promulgated by JEDEC, such as JESD79F for DDR SDRAM, JESD79-2F for DDR2 SDRAM, JESD79-3F for DDR3 SDRAM, JESD79-4A for DDR4 SDRAM, JESD209 for Low Power DDR (LPDDR), JESD209-2 for LPDDR2, JESD209-3 for LPDDR3, and JESD209-4 for LPDDR4. Such standards (and similar standards) may be referred to as DDR-based standards and communication interfaces of the storage devices that implement such standards may be referred to as DDR-based interfaces.

The network interface 206 may be embodied as any hardware, software, or circuitry (e.g., a network interface card) used to connect the mobile device 104 to other devices over a short-range wireless connection or over the network 112, as well as providing the network communication component functions described above. For example, the network interface 206 may be embodied as any communication circuit, device, or collection thereof, capable of enabling communications over the network 112 between the mobile device 104 and other devices. The network interface 206 may be configured to use any one or more communication technology (e.g., wired, wireless, and/or cellular communications) and associated protocols (e.g., Ethernet, Bluetooth®, Wi-Fi®, WiMAX, 5G-based protocols, etc.) to effect such communication. For example, to do so, the network interface 206 may include a network interface controller (NIC, not shown), embodied as one or more add-in-boards, daughtercards, controller chips, chipsets, or other devices that may be used by the mobile device 104 for network communications with remote devices. For example, the NIC may be embodied as an expansion card coupled to the I/O device interface 204 over an expansion bus such as PCI Express.

The I/O device interface 204 allows I/O devices to communicate with hardware and software components of the mobile device 104. For example, the I/O device interface 204 may be embodied as, or otherwise include, memory controller hubs, input/output control hubs, integrated sensor hubs, firmware devices, communication links (e.g., point-to-point links, bus links, wires, cables, light guides, printed circuit board traces, etc.), and/or other components and subsystems to facilitate the input/output operations. In some embodiments, the I/O device interface 204 may form a portion of a system-on-a-chip (SoC) and be incorporated, along with one or more of the processor 202, the memory 210, and other components of the mobile device 104.

The I/O devices 205 may be embodied as any type of I/O device connected with or provided as a component to the mobile device 104. For example, the I/O devices 205 includes an audio I/O 207, which may be embodied as any internal and external audio device for receiving or outputting audio, such as microphone devices, speaker devices, wireless headphones, wired headphones, and the like. Illustratively, the memory 210 includes the app 106, further described herein.

The storage 212 may be embodied as any type of devices configured for short-term or long-term storage of data such as, for example, memory devices and circuits, memory cards, hard disk drives (HDDs), solid-state drives (SSDs), or other data storage devices. The storage 212 may include a system partition that stores data and firmware code for the storage 212. The storage 212 may also include an operating system partition that stores data files and executables for an operating system. As shown, the storage 212 includes application data 214. The application data 214 may be embodied as any type of data generated or maintained by the app 106, further described herein. In an embodiment, the application data 214 may be stored elsewhere from or in addition to the storage 212. For example, the application data 214 may also reside in a storage location managed by the computing device 108.

Referring now to FIG. 3, the app 106, when executed by the mobile device 104, may provide an operational environment 300. Illustratively, the environment 300 of includes a network interface component 302, a data processing component 304, a performance evaluation component 306, a feedback component 308, and an output component 310. The application data 214 includes an audio library 312, audio mapping data 314, configuration data 316, and user data 316.

The illustrative network interface component 302 may be embodied as any hardware, software, and/or circuitry for establishing a network connection with external devices and computing systems. The network interface component 302, for example, may establish a short range wireless connection with the plantar sensor devices 102, as shown, for transmitting and receiving data. The network interface component 302 may implement various communication protocols, such as a UDP, TCP, or WAP, for communicating sensor data and control data between the devices 102 and the app 106.

The illustrative data processing component 304 may be embodied as any hardware, software, and/or circuitry for receiving data captured by each of the plantar sensor devices 102 and formatting the data for further conditioning and analysis. For instance, FIG. 3 further illustrates a sensor layout for a left and right plantar sensor device 102 insole. As previously discussed, the example devices 102 may each include sixteen sensors and an IMU. In an embodiment, each individual sensor (and the IMU) may continuously transmit sensor data (e.g., plantar pressure measurements, total force, acceleration, rotation, elapsed time and step count, foot height) via a network interface on the sensors to the app 106. In some embodiments, the sensors (or network interface associated with the sensor device 102) may simply transmit raw data values, a data type identifier, and an identifier associated with the respective sensor. The data processing component 304 may receive the raw values and accompanying identifying information and calculate underlying measurement data of plantar pressure and the like. The data processing component 304 also transmits the formatted data to other components within the app 106 for further processing and analysis.

The illustrative performance evaluation component 306 may be embodied as any hardware, software, and/or circuitry for evaluating the formatted sensor data and measurements against baseline and target data. The performance evaluation component 306 may be configured to do so in various ways. For example, the performance evaluation component 306 may compare sensor data originating from each of the sensors in the devices 102 against specified thresholds or threshold ranges. The performance evaluation component 306 may also compute deviation measures in the event that a given sensor reading is outside the specified threshold range (or exceeds or falls under a given threshold). As another example, the performance evaluation component 306 may generate a similarity score of a holistic collection of sensor data corresponding to a movement and evaluate that score against a target score to determine whether the score exceeds, falls under, and/or is within a given range of the score.

In an embodiment, the aforementioned thresholds and threshold ranges may be predefined based on a training objective provided for selection by the app 106 to the user (in addition to other characteristics, such as user baseline data). Training objectives may generally be directed to recovery, condition treatment, stability, performance, and the like. Each objective may also be associated with targeted subobjectives, such as easing pressure on a given portion of the foot, joint load distribution, target cadence, gait retraining, target stride, push-off focus, speed, balance, and so on. Each objective or subobjective may have a different threshold configuration for each sensor on the sensor devices 102 to achieve the intended purpose thereof.

The illustrative feedback component 308 may be embodied as any hardware, software, and/or circuitry for generating, retrieving, or modifying audio content to be output as feedback through an audio device connected with the mobile device 104. The feedback component 308 may determine the feedback based on a playback mode provided for selection by the app 106 to the user. One example playback mode may include an audio stream, in which an audio file is provided to a user and altered based on user movement. Another example playback mode may include generated audio, in which a sound (e.g., a chord) is produced in response to touches of the foot of the user (wearing the sensor device 102 insoles) on the ground, in which a combination of sensor inputs may correspond to a given chord. Yet another example playback mode may include variations on the above modes, such as progressively layering auditory feedback in response to user movement (e.g., adding different instruments to a baseline sound).

The feedback component 308 may be configured to retrieve locally stored audio from an audio library 312 to be used for generation and modification. During a training session for a user, the feedback component 308 may retrieve evaluation data form the performance evaluation component 306 and modify the audio based on the data. For example, in the audio stream playback mode, the feedback component 308 may use low pass filtering to muffle a stream of an audio track in the event that user movement deviates from a threshold range. As another example, the feedback component 308 may manipulate audio track data having multiple channels such that each channel is introduced in succession as the user movement progresses through movements within the threshold range.

The output component 310 may be embodied as any hardware, software, and/or circuitry for controlling audio output components connected with the mobile device 104 (e.g., internal speakers, external headphone devices connected with the mobile device 104, etc.). For example, output component 310 may transmit a command to the I/O device interface to cause the audio output device to playback auditory feedback generated by the feedback component 308.

The components 302, 304, 306, 308, and 310 may generate and/or use application 214 in operation. As shown, application data 214 may include an audio library 312, audio mapping data 314, configuration data 316, and user data 318. The audio library 312 may include audio files and assets (e.g., sound recordings, waveforms, music tracks, feedback definitions, Musical Instrument Digital Interface (MIDI) files, etc.) that the app 106 may use in auditory feedback generation, modification, and playback. For example, the feedback component 308 may generate and modify audio retrieved from the audio library 312. The audio mapping data 314 may include mapping specifications of sounds (e.g., chords, channels, layers, and the like) to pressure values at each of the sensors of the plantar sensor devices 102. The configuration data 316 may include control and configuration settings for the app 106 and the plantar sensor devices 102. The user data 318 may include demographic (e.g., gender, race, age, etc.) and bioinformatics data (e.g., height, weight, diagnosed conditions, etc.) associated with a given user. The app 106 may use the user data 318 as a basis for initializing and calibrating the plantar sensor devices 102.

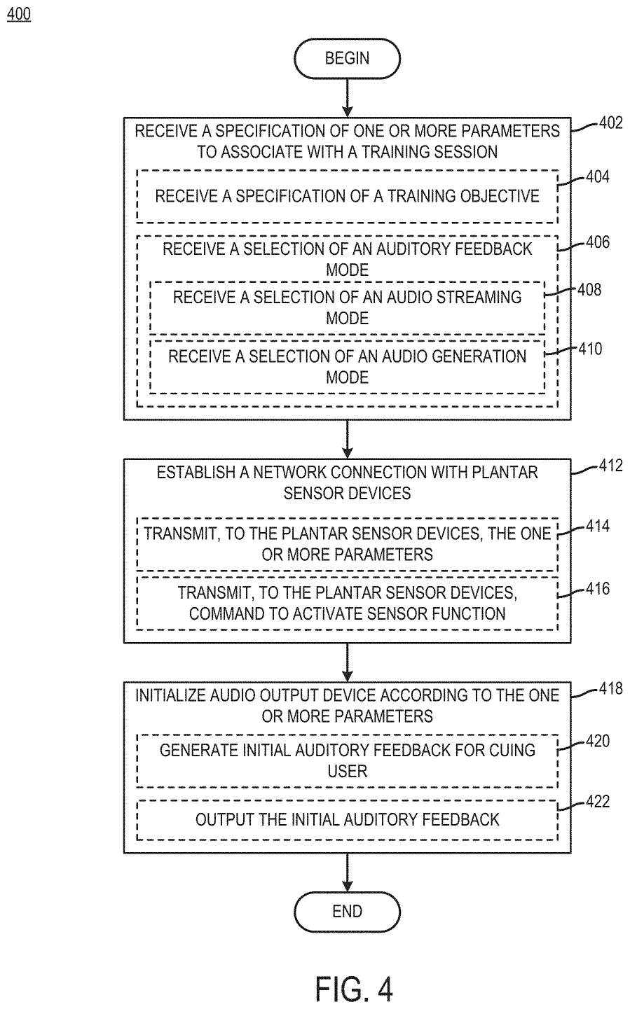

Referring now to FIG. 4, the app 106, in operation, performs a method 400 for initializing a training session (e.g., a walking session, running session, etc.) for providing auditory feedback to a user. As shown, the method 400 begins in block 402, in which the app 106 receives a specification of one or more parameters to associate with a training session. For instance, the app 106 may provide a user interface that allows a user or clinician to specify a variety of settings to apply to the training session. One example of a parameter is shown in block 404, in which the app 106 receives a specification of a training objective (e.g., rehabilitation training for a given body part, stability training, stride optimization, etc.). Another example is shown in block 406, in which the app 106 receives a selection of an auditory feedback mode. In block 408, for instance, the app 106 may receive a selection of an audio streaming mode. Alternatively, in block 410, the app 106 can receive a selection of an audio generation mode. The app 106 may apply the received parameters to the training session.

In block 412, the app 106 establishes a network connection with the plantar sensor devices 102. For example, to do so, the app 106 may initiate a connection request using the BLUETOOTH communication protocol. Upon establishing the connection, in block 414, the app 106 may transmit, to the plantar sensor devices 102 over the connection, the one or more parameters. Further, in block 416, the app 106 may transmit, to the plantar sensor devices 102, a command to activate sensor functions, which in turn causes the plantar sensor devices 102 to begin continuous capture of sensor data.

In block 418, the app 106 initializes the audio output device of the mobile device 104 according to the one or more parameters. The app 106 may retrieve (or generate) an audio file, such as a music track, from the audio library. Further, in block 420, the app 106 may generate, from the audio file, and initial auditory feedback for cuing the user to begin moving for the training session. In block 422, the app 106 may output the initial auditory feedback.

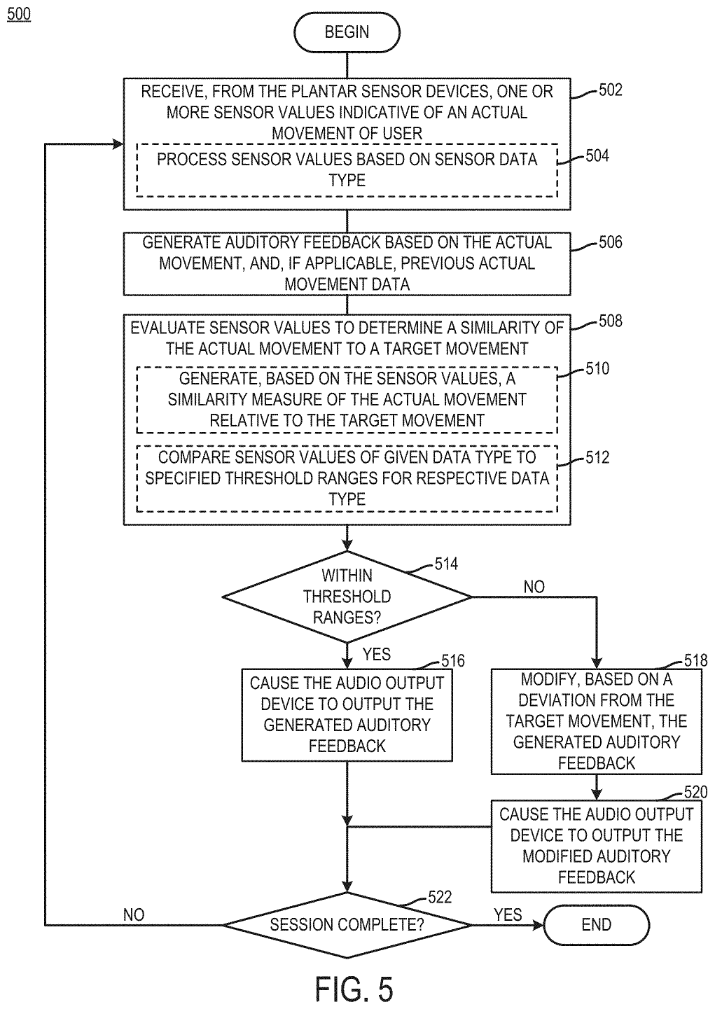

Referring now to FIG. 5, the app 106, in operation, may perform a method 500 for providing continuous and progressive auditory feedback in response to user movement during a training session. In block 502, the app 106 receives, from the plantar sensor devices 102, one or more sensor values indicative of an actual movement of the user. In block 504, the app 106 may process the sensor values, e.g., sorting the data based on data type (e.g., plantar pressure, acceleration, foot height, orientation, stride, etc.). In block 506, the app 106 generates auditory feedback based on the actual movement. For example, the app 106 may identify a portion of a retrieved music track to play in accordance with the actual movement. If applicable (e.g., if the mobile device 104 is already outputting auditory feedback associated with previous movements), the app 106 may base the generated feedback on the previous actual movement data.

In block 508, the app 106 evaluates sensor values to determine a similarity of the actual movement to a target movement. For example, assume the training objective associated with the session corresponds to applying a given pressure on a certain portion of the heel. The target movement may specify one or more thresholds or threshold ranges targeted to achieve such objective and ensure that the actual movement is properly executed. For example, in block 510, the app 106 may generate, based on the sensor values, a similarity measure of the actual movement relative to the target movement. As another example, in block 512, the app 106 may compare sensor values of a given data type to specified threshold ranges for the respective data type.

In block 514, the app 106 may determine whether the sensor data falls within the respective threshold ranges. If so, then in block 516, the app 106 may cause the audio output device of mobile device 104 to output the generated auditory feedback. Otherwise, in block 518, the app 106 may modify, based on a measure of deviation relative to the target movement, the generated auditory feedback. For example, upon determining that a threshold of 50 N exceeded, the app 106 may apply low pass filtering on a currently playing music track to muffle the audio. As another example, the app 106 may deactivate one or more of the audio channels of the underlying music track. As yet another example, the app 106 may generate a chord that is outside of a chord sequence progression being output to the user, resulting in a disharmonic sound. In block 520, the app 106 causes the audio output device to output the modified auditory feedback.

In block 522, the app 106 determines whether the training session is complete (e.g., a timer expires, the audio track completes playback, etc.). If not, then the method 500 returns to block 502. If so, then the method 500 ends.

As a treatment solution, the auditory feedback techniques of the present disclosure may target several modifications and conditions. For example, the auditory feedback may be used to treat medial knee osteoarthritis, in which it is proven that applying more pressure to the medial edge of the foot, and eventually medialized center of pressure, can yield an improved joint load distribution in the knee. Better load distribution can improve pain levels and improve quality of life outcomes. As another example, the techniques are able to treat patients that show a higher pressure on the lateral edge of the foot due to conditions such as ankle fracture, chronic ankle instability, and patellofemoral syndrome (knee pain). The techniques of the present disclosure may also treat conditions or diseases that result in a slower walking speed or shorter stride, such as aging and injuries in the lower limb. As yet another example, the techniques are able to treat conditions that prevent patients from applying more force on their toes on the push-off phase during walking (which is used to project the body forward), such as ankle fracture, multiple sclerosis, and diabetes. Further, through the techniques described herein, a patient afflicted with patellofemoral pain may benefit by reducing pressure during running and achieve higher step frequency. In addition, the techniques may be beneficial to those who suffer from balance impairments (e.g., due to aging, neurological disease, or orthopedic injury). Balance training may be enhanced by using musical audio feedback to better comprehend plantar pressure application and move accordingly to a target specified by a clinician.

As a performance solution, the auditory feedback techniques of the present disclosure may assist a user (e.g., a sports or recreational athlete) in optimizing physical performance. For example, the techniques of the present disclosure may assist the user in better tracking cadence and controlling speed increases.

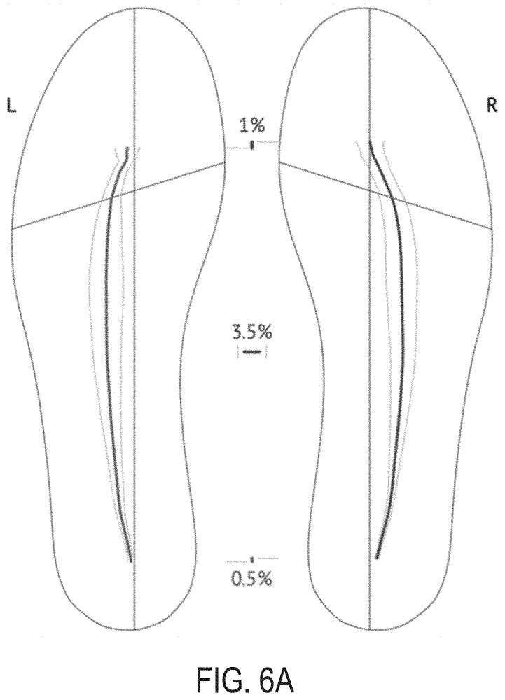

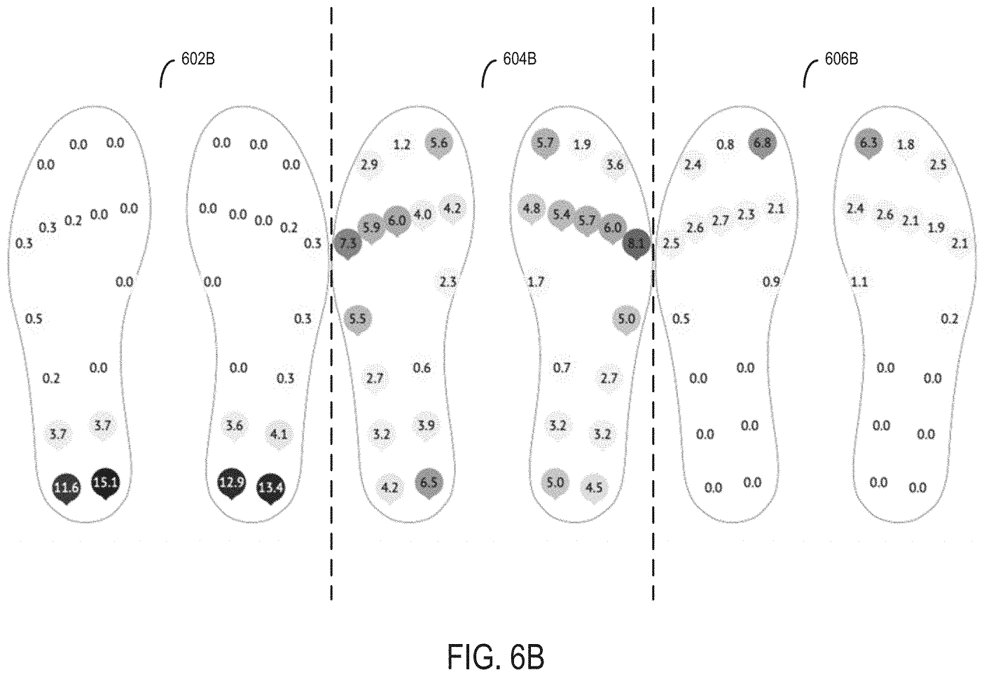

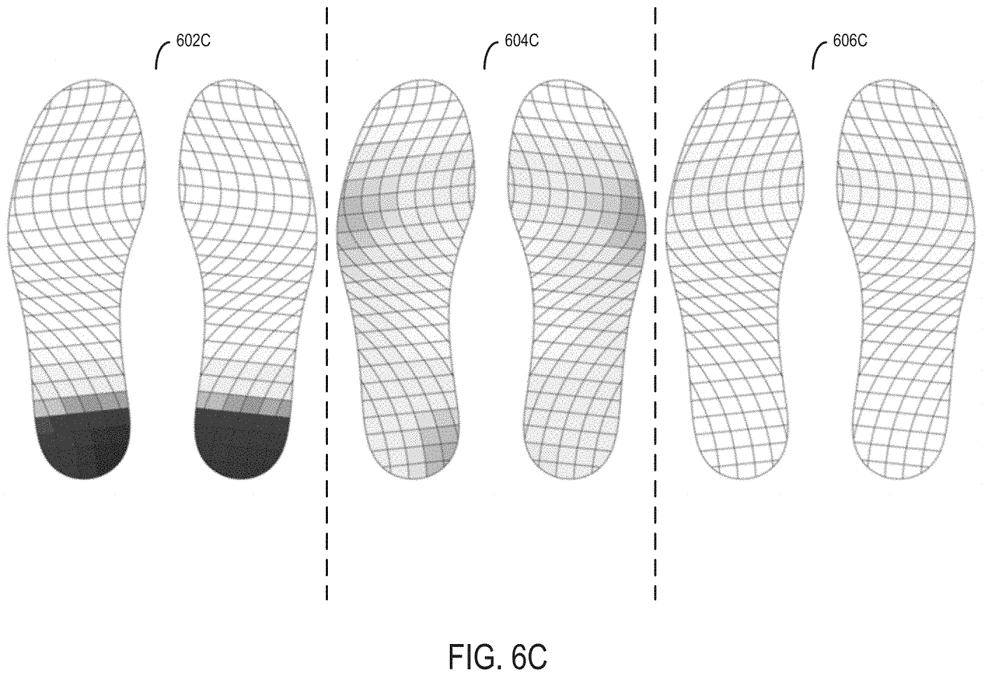

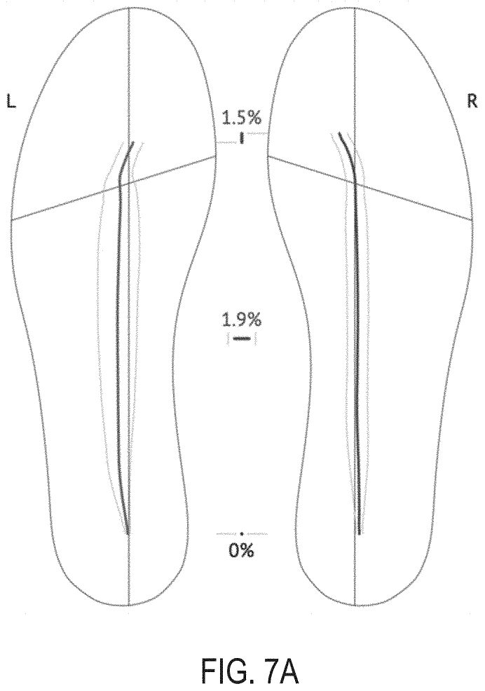

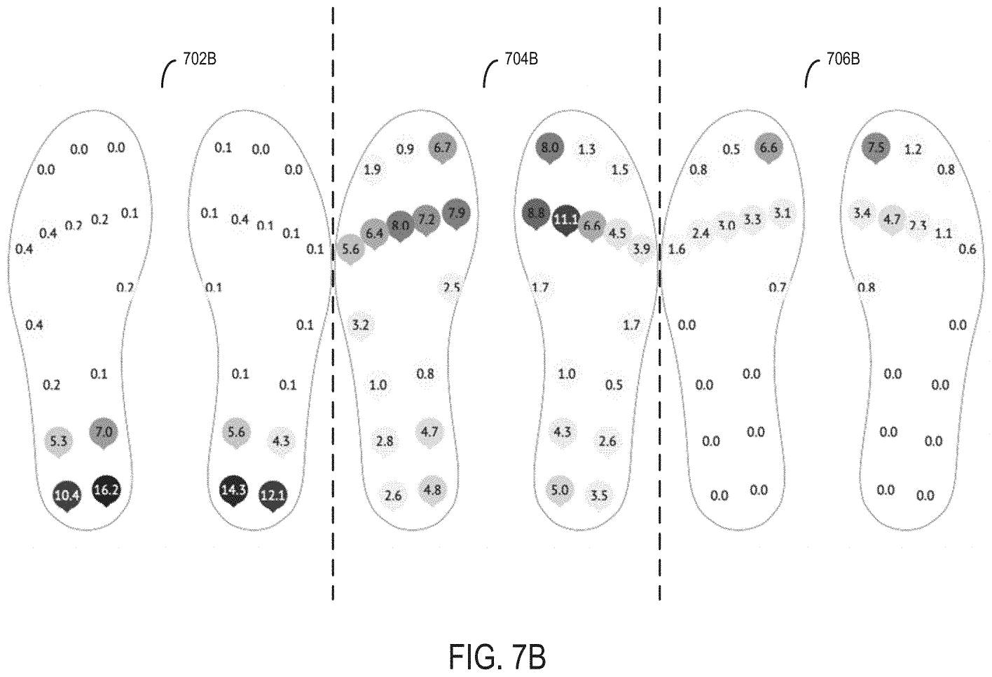

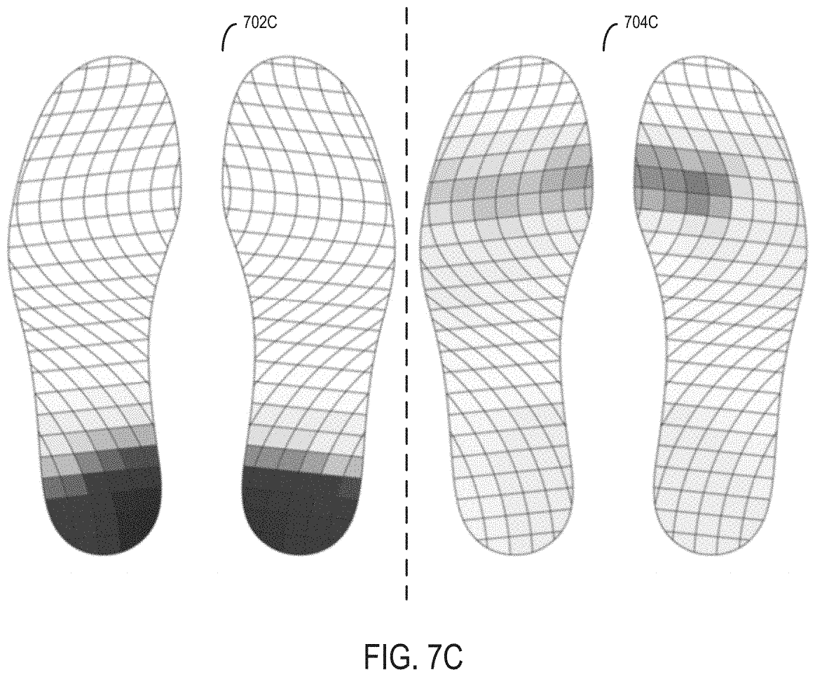

FIGS. 6A-6C and 7A-7C illustrate diagrams pertaining to experimental results demonstrating how a user might change a walking pattern using the techniques of the present disclosure. In the example experiment, the user was tested under two conditions and compared with a baseline. The first condition pertained to providing musical feedback to cue the user to step more with the medial edge of the foot along the walking activity. The second condition pertained to the user receiving different musical feedback and being cued to match the walking tempo to a predetermined tempo (20% above the user's own baseline). Such condition may be beneficial for increasing the speed of a walking activity or running activity (e.g., if the user is aiming to increase physical conditioning).

FIGS. 6A-6C show results averaged for baseline data collection of a three-minute walk (taking place over 339 steps). FIG. 6A illustrates a gait line report of the left and right feet, in which a percentage delta in gait for the frontal, medial, and rear edges of the foot are shown. FIG. 6B illustrates pressure values obtained by the left and right sensor devices 102 at initial contact 602B, mid stance 604B, and terminal stance 606B. FIG. 6C illustrates a pressure distribution, in which the more solid and darker shadings indicate a higher pressure placement at the corresponding region of the foot at initial contact 602C, mid stance 604C, and terminal stance 606C. At baseline, the user had a mean gait (walking) cadence of 55.5 strides per minute.

FIGS. 7A-7C show results averaged for data collected while musical auditory feedback was provided according to the techniques of the present disclosure to cue the user to apply pressure onto the medial side of the foot. FIG. 7A illustrates a gait line report of the left and right feet, in which a percentage delta in gait for the frontal, medial, and rear edges of the foot are shown. FIG. 7B illustrates pressure values obtained by the left and right sensor devices 102 at initial contact 702B, mid stance 704B, and terminal stance 706B. FIG. 7C illustrates a pressure distribution, in which the more solid and darker shadings indicate a higher pressure placement at the corresponding region of the foot at initial contact 702C and mid stance 704C. As demonstrated, average pressure on the medial sensors of the foot demonstrated higher values in comparison to those of the baseline. Further, the gait line of FIG. 7A shows a more centralized line along the walking activity. Further still, compared with the baseline, the user had increased the mean gait cadence to 66.8 strides per minute (a 20.36% increase in cadence).

The embodiment(s) detailed hereinabove may be combined in full or in part, with any alternative embodiment(s) described.

The disclosed systems and methods can be implemented with a computer system, using, for example, software, hardware, and/or a combination of both, either in a dedicated server, integrated into another entity, or distributed across multiple entities. An exemplary computer system includes a bus or other communication mechanism for communicating information, and a processor coupled with the bus for processing information. The processor may be locally or remotely coupled with the bus. By way of example, the computer system may be implemented with one or more processors. The processor may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information. The computer system also includes a memory, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus for storing information and instructions to be executed by processor.

According to one aspect of the present disclosure, the disclosed system can be implemented using a computer system in response to a processor executing one or more sequences of one or more instructions contained in memory. Such instructions may be read into memory from another machine-readable medium, such as data storage device. Execution of the sequences of instructions contained in main memory causes the processor to perform the process steps described herein. One or more processors in a multi-processing arrangement may also be employed to execute the sequences of instructions contained in memory. In alternative implementations, hard-wired circuitry may be used in place of or in combination with software instructions to implement various implementations of the present disclosure. Thus, implementations of the present disclosure are not limited to any specific combination of hardware circuitry and software. According to one aspect of the disclosure, the disclosed system can be implemented using one or many remote elements in a computer system (e.g., cloud computing), such as a processor that is remote from other elements of the exemplary computer system described above.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Numerous modifications to the present disclosure will be apparent to those skilled in the art in view of the foregoing description. Preferred embodiments of this disclosure are described herein, including the best mode known to the inventors for carrying out the disclosure. It should be understood that the illustrated embodiments are exemplary only and should not be taken as limiting the scope of the disclosure.

What is claimed is:

1. A system, comprising:
a plurality of plantar pressure sensors;
an audio output device;
a mobile device comprising a processor and a plurality of instructions, which, when executed by the processor, causes the mobile device to:
receive, from at least one of the plantar pressure sensors, one or more sensor values indicative of an actual movement of a user during a training session,
determine, based on an evaluation of the sensor values, a similarity of the actual movement to a target movement,
modify, as a function of the similarity of the actual movement to the target movement, an auditory feedback to be output, and
cause the audio output device to output the auditory feedback.

2. The system of claim 1, wherein the auditory feedback is one of a plurality of sequential portions of a music audio track.

3. The system of claim 2, wherein the music audio track includes a plurality of audio channels.

4. The system of claim 3, wherein to modify the auditory feedback comprises to activate playback of one of the plurality of audio channels.

5. The system of claim 3, wherein to modify the auditory feedback comprises to deactivate playback of one of the plurality of audio channels.

6. The system of claim 1, wherein the auditory feedback is a generated chord of a chord sequence progression.

7. The system of claim 6, wherein the plurality of instructions further causes the mobile device to:
receive one or more second sensor values indicative of a second actual movement of the user;
modify, as a function of a similarity of the second actual movement to a second target movement, a second auditory feedback, wherein the second auditory feedback is a next chord in the chord sequence progression relative to the generated chord.

8. The system of claim 6, wherein the plurality of instructions further causes the mobile device to:
receive one or more second sensor values indicative of a second actual movement of the user;
modify, as a function of a similarity of the second actual movement to a second target movement, a second auditory feedback, wherein the second auditory feedback is a chord outside the chord sequence progression.

9. The system of claim 1, wherein to determine the similarity of the actual movement to the target movement comprises to generate, based on the one or more sensor values, a similarity measure of the actual movement relative to the target movement.

10. The system of claim 1, wherein the plurality of instructions further causes the mobile device to process the one or more sensor values based on one or more sensor data types.

11. The system of claim 10, wherein to determine the similarity of the actual movement to the target movement comprises to compare sensor values of a given sensor data type to specified threshold ranges for a respective sensor data type.

12. The system of claim 10, wherein the one or more sensor data types comprises at least one of a plantar pressure, total force, acceleration, foot height, stride, or cadence.

13. The system of claim 1, wherein the plurality of instructions further causes the mobile device to generate the auditory feedback based on the actual movement.

14. The system of claim 13, wherein the generation of the auditory feedback is further based on one or more previous actual movements.

15. The system of claim 1, wherein the plurality of instructions further causes the mobile device to receive a specification of one or more parameters to associate with the training session.

16. The system of claim 15, wherein to receive the specification of the one or more parameters comprises to receive a specification of a training objective.

17. The system of claim 16, wherein to receive the specification of the one or more parameters comprises to receive a specification of an auditory feedback mode.

18. The system of claim 17, wherein to receive the selection of the auditory feedback mode comprises to receive a selection of an audio streaming mode.

19. The system of claim 17, wherein to receive the selection of the auditory feedback mode comprises to receive a selection of an audio generation mode.

20. The system of claim 1, wherein the plurality of instructions further causes the mobile device to calibrate the plurality of plantar sensor devices based on demographic and bioinformatics data associated with the user.
